(19) United States
(12) Patent Application Publication
(10) Pub. No.: US 2024/0317661 A1
(43) Pub. Date: Sep. 26, 2024

(54) SYNTHESIS OF HALOGENATED ALKOXYETHANE

(71) Applicant: COMMONWEALTH SCIENTIFIC AND INDUSTRIAL RESEARCH ORGANISATION, Acton (AU)

(72) Inventors: John TSANAKTSIDIS, Acton (AU); Cecily ELDRIDGE, Acton (AU); Scott COURTNEY, Scoresby (AU)

(21) Appl. No.: 18/571,585

(22) PCT Filed: Jun. 17, 2022
(86) PCT No.: PCT/AU2022/050616

§ 371 Date: Dec. 18, 2023

(30) Foreign Application Priority Data
Jun. 18, 2021 (AU) 2021901842

(51) Int. Cl. C07C 41/06; C07C 41/42

(52) U.S. Cl. CPC C07C 41/06; C07C 41/42

(57) ABSTRACT

A process for continuous preparation of halogenated alkoxyethane of general formula XClHC—CF₂OR, where X is —Cl or —F and OR is C₁₋₄ alkoxy, the process comprising a step of introducing in a plate reactor reaction components comprising (i) a compound of general formula XClC═CF₂, (ii) a base, and (iii) a C₁₋₄ alkanol, wherein a) the plate reactor comprises a fluidic module defining one or more fluidic path(s) through which the reaction components flow as a reaction mixture, and b) the halogenated alkoxyethane is formed at least upon the reaction components mixing, with the so formed halogenated alkoxyethane flowing out of the plate reactor in a reactor effluent.

### FIELD OF THE INVENTION

The present invention relates in general to continuous preparation of halogenated alkoxyethane, and in particular to a process for continuous preparation of halogenated alkoxyethane of general formula XClHC—CF₂OR, where X is —Cl or —F and OR is C₁₋₄ alkoxy.

### BACKGROUND

Halogenated alkoxyethane compounds constitute a significant fraction of present day active pharmaceutical ingredients, not to mention agrochemicals, dyes, flame-retardants, and imaging agents.

Synthesis of halogenated alkoxyethane compounds for use as active pharmaceutical ingredients requires reproducible pharmaceutical grade compounds. Conventionally, halogenated alkoxyethane compounds are produced through batch procedures.

However, the product quality per batch can be variable and the procedures can require the use of costly high-pressure equipment. Current batch procedures can be plagued by poor and inhomogeneous reagent mixing and necessitate long reaction times for relatively low conversion yields. As a result, conventional batch synthesis of halogenated alkoxyethane compounds can require costly post-processing purification procedures to ensure that a pharmaceutical grade compound is produced at a commercially relevant scale.

In contrast to conventional batch procedures, continuous production using semi-batch or semi-continuous arrangements are appealing in that they can potentially afford higher yield relative to conventional batch procedures. However, for the production of halogenated alkoxyethane specifically, existing semi-batch or semi-continuous arrangements struggle to offer effective management of toxic and corrosive intermediates and by-products, and do not fully address the challenges of conventional batch processes in terms of thermal control, safety, waste management, high reaction times, and low conversion yield.

Accordingly, there remains an opportunity to ameliorate problems and limitations associated with conventional procedures for the synthesis of halogenated alkoxyethane compounds.

### SUMMARY OF THE INVENTION

The present invention relates to a process for continuous preparation of halogenated alkoxyethane of general formula XClHC—CF₂OR, where X is —Cl or —F and OR is C₁₋₄ alkoxy, the process comprising a step of introducing in a plate reactor reaction components comprising (i) a compound of general formula XClC═CF₂, (ii) a base, and (iii) a C₁₋₄ alkanol, wherein
a) the plate reactor comprises a fluidic module defining one or more fluidic path(s) through which the reaction components flow as a reaction mixture, and
b) the halogenated alkoxyethane is formed at least upon the reaction components mixing, with the so formed halogenated alkoxyethane flowing out of the plate reactor in a reactor effluent.

By the present invention, the reaction components can be continuously introduced into the plate reactor and converted therein into a reactor effluent containing the targeted halogenated alkoxyethane. The effluent continuously flows out of the reactor and is available for further processing and/or purification, if needed. The continuous nature of the process advantageously enables halogenated alkoxyethane to be produced in commercial quantities.

In its simplest configuration, a fluidic module for use in the plate reactor would have a single fluidic path connecting a fluidic inlet and a fluidic outlet of the fluidic module. In more complex configurations, a fluidic module may have multiple fluidic paths connecting one or more fluidic inlets and one or more fluidic outlets of the fluidic module. Said multiple fluidic paths may merge, effecting mixing of their respective fluids.

In some embodiments, the plate reactor comprises multiple fluidic modules. Said modules may be connected in series, such that a given fluidic outlet of a given module is in fluid communication with a given fluidic inlet of a subsequent module to provide a continuous fluidic path across all modules. In some embodiments, the plate reactor comprises multiple fluidic modules connected in parallel. In some embodiments, the plate reactor comprises multiple fluidic modules, some of which are connected in series and some in parallel.

The one or more fluidic path(s) in a fluidic module may have any dimension and design that are conducive to the reagent components flowing as a reaction mixture through the reactor. From the design standpoint, the one or more fluidic path(s) may be in the form of channels, at least a portion of which has constant cross-section along the main axis, and/or channels at least a portion of which has variable cross-sectional area along their main axis.

In the process of the invention, the halogenated alkoxyethane forms at least upon the reaction components mixing. The reaction is exothermic and reaction heat can be continuously extracted by any means known to the skilled person in the context of plate reactors. Heat extraction may achieved by controlling the temperature of each fluidic module. In some embodiments, the fluidic modules are at a temperature of from about −15° C. to about 45° C. In some embodiments, the fluidic modules are at a temperature of from about −10° C. to about 25° C. The proposed temperature range has been observed to be particularly advantageous for the high-yield production of methoxyflurane.

In some embodiments, the reaction components flow as a reaction mixture through the one or more fluidic path(s) at an average flow rate of about 1-15 ml/min. As a skilled person would appreciate, specific flow rates would be obtained by suitable combinations of design and process parameters, which may include the dimensional design of the one or more fluidic path(s), the operational temperature, and the overpressure along the entire fluidic path in the plate reactor.

Flow along the one or more fluidic path(s) is characterised by a certain degree of fluidic resistance. Said fluidic resistance can be quantified in terms of pressure drop between an inlet and an outlet of the one or more fluidic path(s). In turn, for a given design of the one or more fluidic path(s) the pressure drop is proportional to the flow rate of the reaction mixture along the one or more fluidic path(s). Typically, the pressure drop would be such that the reaction mixture can effectively flow along the one or more fluidic path(s).

Pressure within the one or more fluidic path(s) can be regulated by any means known to a skilled person. For example, the pressure may be regulated by ways of a backpressure valve located downstream of the reactor, a pressure transducer (PT) and/or a back pressure regulatory (BPR) system.

It will be understood that the operational characteristics of the fluidic modules in the plate reactor of the invention (e.g. pressure, flow rate, dimensions, etc.) afford industrial production of the halogenated alkoxyethane. This effectively places the plate reactor within the class of industrial reactors, for example in opposition to micro-fluidic reactors.

The specific design of the one or more fluidic path(s) and process conditions (e.g. temperature and pressure drop) afford fast and thorough mixing of the reaction components, leading to significant improvement over conventional procedures in terms of reaction time and conversion yield.

In addition, the one or more fluidic path(s) provide a much more controlled environment for reaction relative to conventional systems used in batch processes, making the plate reactor of the invention inherently safer to operate and affording the production of a purer product relative to conventional apparatuses. In that context, extreme conditions of temperature and pressure are readily implemented in the reactor of the invention to boost chemical reactivity, yet keeping full control on process parameters.

Thus, high reaction selectivity and enhanced safety can be achieved even for very fast and highly exothermic reactions involved in the formation of the target halogenated alkoxyethane. The excellent heat and mass transfer characteristics afforded by the one or more fluidic paths, together with the fact that the reaction is resolved along the length of the reaction channel, enables a precise control of the residence time of intermediates or products by a thermal or chemical quench of the solution.

Further, the controlled environment for reaction afforded by small-section fluidic paths ensures that formation of hazardous chemicals can be easily controlled. Toxic substances can be readily quenched in line, thus avoiding any undesired exposures and significantly enhancing process safety.

The process of the invention is also particularly advantageous for the production of commercially relevant halogenated alkoxyethane compounds.

For example, the compound of general formula XClC═CF₂ may be Cl₂C═CF₂. In those instances, the process of the invention allows for the efficient and scalable production of halogenated alkoxyethane compounds such as methoxyflurane (Cl₂HC—CF₂OCH₃), which can be obtained when the C₁₋₄ alkanol is methanol. Given its high reaction yield, the process can afford facile and large-scale synthesis of pharmaceutical grade methoxyflurane.

For the production of methoxyflurane, the temperature of the fluidic module (or multiple connected fluidic modules) may be advantageously controlled to a temperature between about −10° C. to about 25° C. In those instances, the reaction mixture may be made to flow across the plate reactor at a flow rate between about 15 ml/min and about 100 ml/min.

Those embodiments can provide an advantageous compromise between good thermal control and safety, low reaction times, high conversion yields, and high scale-up potential for the high throughput production of pharmaceutical grade methoxyflurane.

In some embodiments, the compound of general formula XClC═CF₂ is FClC═CF₂. In those instances, the process of the invention affords efficient and scalable production of ClFHC—CF₂OCH₃, which can be obtained when the C₁₋₄ alkanol is methanol. The possibility to produce highly pure and high amounts of ClFHC—CF₂OCH₃ can be particularly advantageous, since that compound is a known precursor in the synthesis of 2-chloro-1,1,2,-trifluoroethyl-difluoromethyl ether (enflurane).

Further aspects and embodiments of the invention are discussed in more detail below.

### BRIEF DESCRIPTION OF THE DRAWINGS

The invention will also be described herein with reference to the following non-limiting drawings in which:

FIG. 1 shows a first embodiment fluidic module of a plate reactor for use in the process of the invention,

FIG. 2 shows a second embodiment fluidic module of a plate reactor for use in the process of the invention,

FIG. 3 shows a third embodiment fluidic module of a plate reactor for use in the process of the invention,

FIG. 4 shows a fourth embodiment fluidic module of a plate reactor for use in the process of the invention,

FIG. 5 shows the ¹H Nuclear magnetic resonance (NMR) trace recorded on a product fraction extracted at the reactor outlet,

FIG. 6 shows the ¹³C NMR trace recorded on a product fraction extracted at the reactor outlet, and

FIG. 7 shows the ¹⁹F NMR trace recorded on a product fraction extracted at the reactor outlet.

### DETAILED DESCRIPTION OF THE INVENTION

The process of the invention is one for continuous preparation of halogenated alkoxyethane of general formula XClHC—CF₂OR, where X is —Cl or —F and OR is C₁₋₄ alkoxy.

As used herein, the expression “C₁₋₄ alkoxy” denotes a straight chain or branched alkoxy group having from 1 to 4 carbons. Examples of straight chain and branched alkoxy include methoxy, ethoxy, n-propoxy, isopropoxy, n-butoxy, sec-butoxy, and t-butoxy.

In some embodiments, X is —Cl and OR is a methoxy group, in which case the halogenated alkoxyethane has a formula Cl₂HC—CF₂OCH₃ (methoxyflurane).

In some embodiments, X is —F and OR is a methoxy group, in which case the halogenated alkoxyethane has a formula FClHC—CF₂OCH₃. Such compound is a known precursor for the synthesis of 2-chloro-1,1,2,-trifluoroethyl-difluoromethyl ether (enflurane).

The process of the invention is one for the continuous preparation of halogenated alkoxyethane, and is based on the use of a plate reactor. By the preparation being “continuous” is meant that the halogenated alkoxyethane forms continuously as the reagent components are mixed and flow through the one or more fluidic path(s). As such, the so-formed halogenated alkoxyethane can be collected from the effluent that exits the plate reactor continuously.

The plate reactor used in the process of the invention comprises one or more fluidic path(s). The expression “fluidic path” is used herein to mean a continuous fluidic line along which a fluid can flow. In the context of a plate reactor, said fluidic line may be visualised as a channel placing an inlet and an outlet of a fluidic module in fluid communication. Accordingly, a fluidic path may have the form of a channel embedded within a solid plate, for example a fluidic module of the kind described herein.

Accordingly, by a “plate reactor” is meant a reactor comprising at least one fluidic module, each module having at least one fluidic path(s) connecting one or more fluidic inlet(s) with one or more a fluidic outlet(s) of the module. In a typical configuration, the plate reactor is made by at least one or more planar fluidic module, each defining one or more fluidic path(s) on a plane.

In its simplest configuration, a fluidic module would have a single fluidic path providing fluid connection between one fluidic inlet and one fluidic outlet. Multiple fluidic modules can be connected together such that a given fluidic outlet of a given module is connected with a given fluidic inlet of the subsequent module to provide a continuous fluidic path across all modules. Said connection may be achieved by means of appropriate fluidic connections known to a skilled person (e.g. tubing, etc.).

Provided the halogenated alkoxyethane forms, the plate reactor may comprise any number of fluidic modules connected to provide the one or more fluidic path(s).

In some embodiments, the plate reactor comprises one fluidic module.

In some embodiments, the plate reactor comprises at least two fluidic modules. For example, the plate reactor may comprise 3, 4, 5, 6, 7, 8, 9, or 10 fluidic modules. In some embodiments, the plate reactor comprises between 2 and 10 fluidic modules. For example, the plate reactor may comprise 5 fluidic modules.

When the plate reactor comprises multiple connected fluidic modules, the fluidic modules may be connected in series, in parallel, or in a combination of series and parallel. This makes the scale up to large production quantities relatively straight forward. As a result, scale-up can be performed with minimal to no re-optimisation of the reaction conditions, since they remain unchanged within each fluidic module. In this context, it can be more effective and efficient to merely “number-up” the fluidic modules to produce a given quantity of halogenated alkoxyethane compared with developing a single macro-fluidic path to produce the same amount of halogenated alkoxyethane. While a process in accordance with the present invention can be performed to produce small quantities of halogenated alkoxyethane (e.g. fraction of grams per day) by using one fluidic module, multiple fluidic modules can be readily connected to produce more commercially relevant amounts of halogenated alkoxyethane (e.g. from several grams to several kilos per day), yet maintaining identical standards of safety, product purity, reaction time, reaction yield, and safety.

The plate reactor of the invention would be designed to enable (i) continuous introduction of the reaction components into the fluidic path(s) through which they flow as a reaction mixture, and (ii) continuous flow out of the reactor of an effluent containing the halogenated alkoxyethane.

Provided the reaction components flow through the one or more fluidic path(s) as a reaction mixture, there is no particular limitation as to where the components are mixed together relative to the one or more fluidic path(s).

For instance, the reaction components may be mixed together to form the reaction mixture prior to said mixture being introduced into the one or more fluidic path(s).

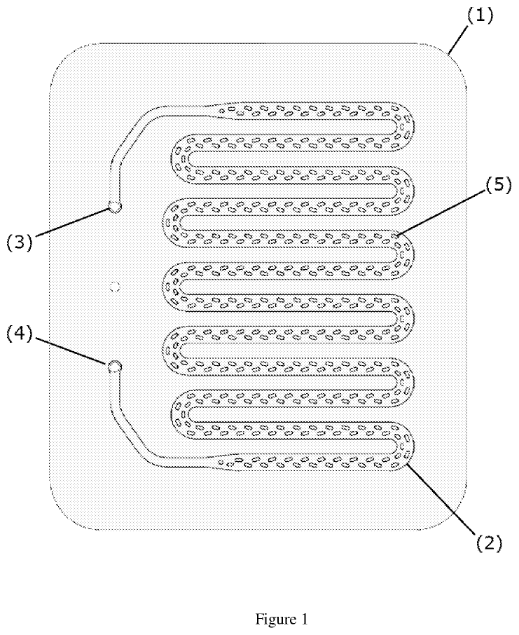

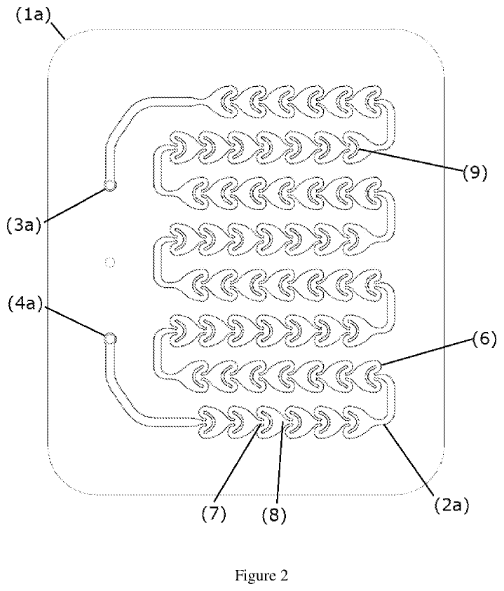

Accordingly, in some embodiments, the reaction components are mixed to form the reaction mixture upstream of the one or more fluidic path(s), and the reaction mixture is subsequently introduced into the one or more fluidic path(s). In those instances, the fluidic modules making the reactor may be characterised by one or more discrete non-intersecting fluidic paths along which the reaction mixture flows across all modules. In some embodiments, a fluidic module of the plate reactor comprises a single fluidic path connecting a fluidic inlet with a fluidic outlet of the module. Examples of such modules are shown in FIGS. 1-2. Multiple modules may be connected to provide a singular fluidic path connecting an inlet and an outlet of the plate reactor.

Alternatively, in some preferred arrangements the reaction components may be introduced into discrete fluidic paths, for example through corresponding dedicated inlets, and made to mix within the module by designing the fluidic paths so that they merge.

Accordingly, in some preferred embodiments, the reaction components are introduced into the plate reactor through distinct inlets. In those instances, a fluidic module of a series of modules forming the reactor (or the only module forming the reactor) would have merging fluidic paths designed to induce mixing of the reaction components.

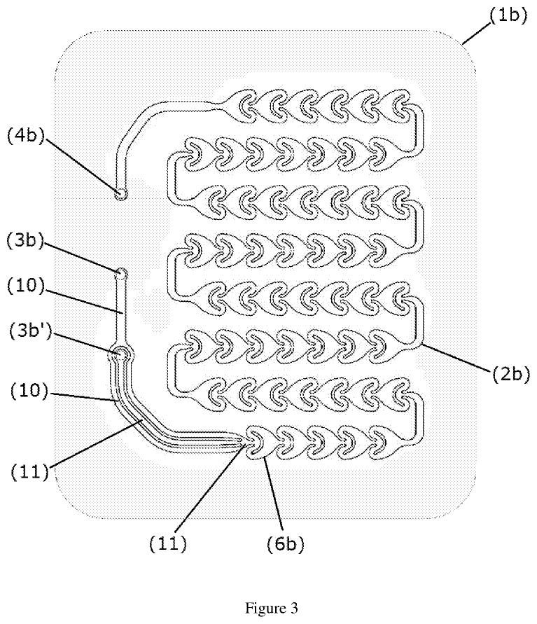

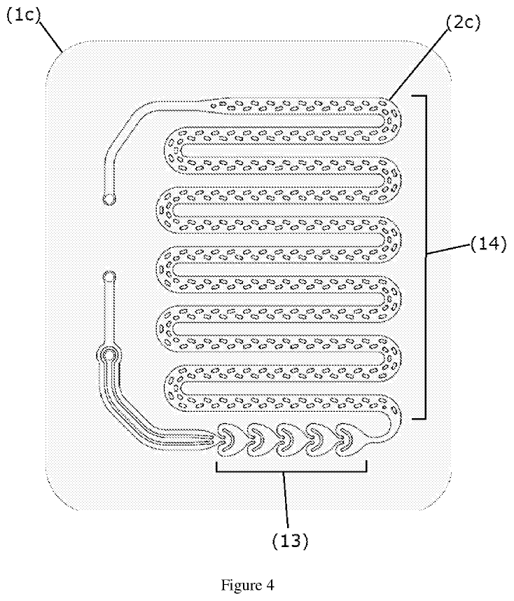

In some preferred embodiments, a fluidic module comprises at least two fluidic inlets originating corresponding fluidic paths that merge such that fluid flowing from each fluidic inlet mix together before reaching a fluidic outlet of the module. Examples of such modules are shown in FIGS. 3 and 4. In those instances, the reactor may comprise one such module, or multiple modules comprising one such module (e.g. the first module of a series).

The one or more fluidic path(s) may have any design that is conducive to the targeted halogenated alkoxyethane forming.

In some embodiments, the fluidic module comprises a fluidic path in the form of a channel at least a portion of which has constant cross-sectional area along the direction of flow. In those instances, opposing internal walls of the channels are essentially parallel relative to one another.

In some embodiments, at least a portion of the one or more fluidic path(s) present as channels having a square or rectangular internal cross-section geometry with constant cross-sectional area along the direction of flow. The average internal diagonal of such a fluidic path may range between about 1 and about 12 mm. The average internal diagonal of a fluidic path with square or rectangular cross-section may typically be greater than or equal to 0.2 mm but less than 12 mm (and including any integer there between, and/or fraction thereof, for example, 0.2 mm, 0.3 mm, 0.4 mm, 0.5 mm, and so on). In one embodiment, the average internal diagonal is greater than or equal to 2 mm but less than or equal to 10 mm. In one embodiment, the average internal diagonal is greater than or equal to 2 mm but less than or equal to 8 mm. In some embodiments, the average internal diagonal is about 6 mm. Those dimensions provide a particularly advantageous combination of effective mixing of the reaction mixture and specific surface area for effective thermal control. For example, fluidic path(s) of any of those sizes are sufficiently large to accommodate a static mixer of the kind described herein, yet provide an adequately large specific surface area for effective thermal control. As a result, the reactor can be operated to provide particularly high yields of halogenated alkoxyethane. The resulting reactor represents therefore an advantageous platform for scaled-up production of pharmaceutical grade halogenated alkoxyethane.

FIG. 1 shows an embodiment fluidic module (1) having a fluidic path (2) a main portion of which has constant cross-sectional area along the direction of flow. The main portion of fluidic path (2) presents as a channel having a square or rectangular internal cross-section geometry, depending on the vertical size of the channel (i.e. perpendicular to the view plane). Module (1) presents inlet/outlet ports (3, 4) through which fluid enters/exits fluidic path (2). The embodiment module of FIG. 1 is suitable for the flow of reaction components that have been mixed upstream of the module, which can flow through fluidic path (2) as a reaction mixture. Static mixers in the form of flat baffles (5) are located along the fluid path to assist with the mixing of the reaction components as the reaction mixture flows though the fluidic path.

In some embodiments, the one or more fluidic path(s) are in the form of channels at least a portion of which presents variable cross-sectional area along the direction of flow.

For example, the channels may present a cross-sectional area characterised by multiple minima and multiple maxima alternating along the direction of flow. As a result, the one or more fluidic path(s) present periodic constrictions along the direction of flow, which assist in generating oscillatory flow. By “oscillatory flow” is meant that the fluid is oscillated in the axial direction of the one or more fluidic path(s) such that it flows along the fluidic path(s) at alternating flow rates. This results in an efficient mixing mechanism where fluid moves from the walls to the centre of the path(s) in an alternating manner based on the frequency of the alternating cross-section restrictions and expansions, and relative spacing of the alternating restrictions and expansions.

In some embodiments, the one or more fluidic path(s) define successive chambers, each with a nozzle-like entrance and a narrowing exit. A chamber of said successive chambers may be nested with a next-succeeding chamber such that the narrowing exit of the one chamber forms the nozzle-like entrance of the next adjacent succeeding chamber. This configuration can be particularly advantageous in that it can provides a tortuous path for fluid flow, further contributing to the mixing of the reaction components. An example of said channel design is shown in FIG. 2.

FIG. 2 shows an embodiment fluidic module (1a) of a plate reactor for use in the process of the invention. The module (1a) defines a fluidic path (2a) between fluidic inlets/outlets (3a, 4a). The fluidic path (2a) defines successive chambers (6), each with a nozzle-like entrance (7) and a tapered exit (8). Tapered exit (8) of each chamber (6) forms the nozzle-like entrance of the next adjacent succeeding chamber. In the depicted module, the exit of each chamber (6) is nested within the successive chamber. In the embodiment, each chamber (6) is provided with an internal curved static baffle (9) that can deflect fluid flow entering the chamber and force it to follow the curved side surfaces of the chamber, which taper into the exit (8) of each chamber. The embodiment module of FIG. 2 is suitable for the flow of reaction components that have been mixed upstream of the module, and that flow through fluidic path (2a) as a reaction mixture.

FIG. 3 shows a variant of the embodiment module of FIG. 2. In the module (1b) of FIG. 3, discrete inlets (3b, 3b′) originate two separate channels (10, 11) that merge at mixing point (11) to form nozzle-like entrance of the first chamber (6b). The remainder of fluidic path (2b) is similar to that of the module of FIG. 2. The embodiment module of FIG. 3 is suitable for the mixing of two input streams into one stream that flows through fluidic path (2b) and exits module (1b) at outlet (4b). For example, module (1b) may be used to mix a pre-formed base/alkanol solution with the XClC═CF₂ compound to form the reaction mixture that flows through fluidic path (2b). The pre-formed base/alkanol solution may be introduced through inlet (3b) and the XClC═CF₂ compound through inlet (3b′). Alternatively, the pre-formed base/alkanol solution may be introduced through inlet (3b′) and the XClC═CF₂ compound through inlet (3b).

In some embodiments, the one or more fluidic path(s) have a design that is a combination of the designs described herein. For example, the one or more fluidic path(s) may alternate sections of constant cross-sectional area along the direction of flow and sections of variable cross-sectional area along the direction of flow. The sections of constant cross-sectional area and sections of variable cross-sectional area along the direction of flow may be of the kind described herein.

FIG. 4 shows an embodiment module (1c) having a fluidic path (2c) that combines a section (13) of variable cross-sectional area of the kind shown in FIGS. 2-3 with a section (14) of constant cross-sectional area of the kind shown in FIG. 1.

The fluidic modules, for example of the kind depicted in FIGS. 1-4, may have any size that is conducive to effective production of the halogenated alkoxyethane. For example, a fluidic module may have a side dimension of at least about 100 mm, at least about 250 mm, at least about 500 mm, or at least about 750 mm. In some embodiments, a fluidic module has a side dimension of from about 100 mm to about 1 m, for example from about 100 mm to about 750 mm, from about 100 mm to about 500 mm, or from about 100 mm to about 250 mm. In some embodiments, the fluidic module(s) has/have a square or rectangular shape with dimensions from about 100×100 mm to about 750×750 mm. In some embodiments, the fluidic module(s) has/have dimensions of about 150×120 mm, about 300×250 mm, about 450×300 mm, about 600×400 mm, or about 700×500 mm.

In the process of the invention, the reaction mixture may flow through the one or more fluidic path(s) at any flow rate that is conducive to generation of the halogenated alkoxyethane. In some embodiments, the reaction mixture flows through the one or more fluidic path(s) at a flow rate of at least about 1 ml/min. For example, the reaction mixture may flow through the one or more fluidic path(s) at a flow rate of at least about 5 ml/min, at least about 25 ml/min, at least about 50 ml/min, at least about 100 ml/min, at least about 250 ml/min, at least about 500 ml/min, at least about 750 ml/min, at least about 1 L/min, at least about 2 L/min, at least about 4 L/min, or at least about 8 L/min.

The one or more fluidic path(s) may provide for any internal volume conducive to generation of the halogenated alkoxyethane. For avoidance of doubt, by “internal volume” of the one or more fluidic path(s) is meant the volume of the internal cavity of the fluidic path(s) through which the reaction components flow as a reaction mixture. In other words, the “internal volume” of the one or more fluidic path(s) corresponds to the total volume of fluid present in the fluidic path(s) at any given time, when the reactor is in operation.

In some embodiments, the one or more fluidic path(s) has/have a total internal volume of at least about 5 ml, at least about 10 ml, at least about 25 ml, at least about 50 ml, at least about 100 ml, at least about 250 ml, at least about 500 ml, at least about 750 ml, at least about 1 L, at least about 1.5 L, or at least about 2 L. For example, the one or more fluidic path(s) may have a total internal volume in the range of 10 ml to 2 L, for example less than or equal to 1 L (and including any integer there between, and/or fraction thereof, for example, 100 ml, 100.1 ml, etc.). In one embodiment, the one or more fluidic path(s) has/have a total internal volume greater than or equal to 10 ml but less than or equal to 1 L. For example, the one or more fluidic path(s) may have a total internal volume greater than or equal to 10 ml but less than or equal to 500 ml. In one embodiment, the one or more fluidic path(s) has/have a total internal volume of greater than or equal to 10 ml but less than or equal to 100 ml.

The volumetric residence time of fluid flowing through the one or more fluidic path(s) can be determined by the ratio of the total internal volume of the fluidic path(s) to the flow rate of the fluid flowing through the fluidic path(s). In turn, the latter may be determined by the sum of the flow rate of all reagent component lines converging into the one or more fluidic path(s). In the process of the invention, the plate reactor may be operated to obtain any residence time of fluid flowing through the one or more fluidic path(s) that is conducive to generation of the halogenated alkoxyethane.

For example, the plate reactor may be operated to provide a residence time of less than about 250 minutes. In some embodiments, the plate reactor is operated to provide a residence time of less than about 200 minutes, less than about 100 minutes, less than about 50 minutes, less than about 25 minutes, less than about 20 minutes, less than about 15 minutes, less than about 10 minutes, less than about 5 minutes, less than about 2.5 minutes, less than about 2 minutes, or less than about 1 minute. In some embodiments, the plate reactor is operated to provide a residence time of from about 1 minute to about 5 minutes.

For avoidance of doubt, it will be understood that irrespective of the form in which the one or more reagent compound(s) are provided, they flow through the one or more fluidic path(s) as a liquid reaction mixture. Accordingly, the present invention may be also said to provide a process for continuous preparation of halogenated alkoxyethane of general formula XClHC—CF₂OR, where X is —Cl or —F and OR is C₁₋₄ alkoxy, the process comprising a step of introducing in a plate reactor reaction components comprising (i) a compound of general formula XClC═CF₂, (ii) a base, and (iii) a C₁₋₄ alkanol, wherein (a) the plate reactor comprises a fluidic module defining one or more fluidic path(s) through which the reaction components flow as a liquid reaction mixture, and (b) the halogenated alkoxyethane is formed at least upon the reaction components mixing, with the so formed halogenated alkoxyethane flowing out of the plate reactor in a reactor effluent.

In some embodiments, the halogenated alkoxyethane is formed by cooling the reaction mixture to a temperature of down to about −15° C. For example, the reaction mixture may be cooled to a temperature of down to about −10° C., down to about −5° C., down to about −2.5° C., down to about −1° C., down to about 0° C., down to about 5° C., down to about 10° C., or down to about 25° C. In some embodiments, the halogenated alkoxyethane is formed at a temperature from 0° C. to 25° C. For example, the halogenated alkoxyethane may be formed at a temperature of about 10° C.

The temperature of any of the reagent compounds may also be controlled to a desired value before they are mixed to form the reaction mixture. For instance, the base and/or the alkanol may be used at room temperature. In some embodiments, the base and the alkanol are provided as a base/alkanol solution. Said base/alkanol solution may be used at a temperature below 15° C., for example below 10° C., or between 0° C. and 15° C. In some embodiments, the XClC═CF₂ compound is used at room temperature. In some embodiments, the XClC═CF₂ compound is used at a temperature below 15° C., for example below 10° C., or between 0° C. and 15° C.

Accordingly, in some embodiments one or more reagent compound(s) are cooled prior to being mixed to form the reaction mixture, such that the one or more reagent compound(s) is/are in liquid form when the reaction mixture forms. Cooling any of the reagent components may be necessary to ensure they are used in liquid form in the plate reactor. This may be achieved by any means known to the skilled person. For example, reservoirs of either or both the base/alkanol solution and the XClC═CF₂ compound may be temperature controlled. In some embodiments, either or both the base/alkanol solution and the XClC═CF₂ compound are provided in corresponding temperature controlled reservoir. Such temperature control may be achieved by cooling strategies of the kind described herein (e.g. cooling jacket, a heat exchanger, or a combination thereof). Alternatively, or at the same time, cooling of one or more reagent component(s) may be achieved by a temperature controlled reservoir pump, for example a pump provided with a cooling system of the kind described herein (e.g. cooling jacket, a heat exchanger, or a combination thereof).

As used herein, “room temperature” refers to ambient temperatures that may be, for example, between 10° C. to 40° C. but is more typically between 15° C. to 30° C. For example, room temperature may be a temperature between 20° C. and 25° C.

The plate reactor in the process of the invention may be operated at any pressure conducive to generation of the halogenated alkoxyethane. The process of the invention the reaction components may flow through the one or more fluidic path(s) at a pressure such that the reaction mixture is kept in liquid form. For example, in the process of the invention, the reaction components may flow through the one or more fluidic path(s) at a pressure of about 1,250 kPa (gauge pressure).

The internal walls of the one or more fluidic path(s), which would be in contact with the reaction components and corresponding mixture, may be made of a material that is chemically inert to the reaction components, the halogenated alkoxyethane, and any reaction intermediate or by-product. In that regard, said material may be the same material the fluidic module is made of. Further, said material should be of suitable strength and structural integrity to withstand the flow rate pressure(s) and volume(s) of fluid passing through it.

In some embodiments, the one or more fluidic path(s) have an internal surface wall made of a metal, an alloy, a ceramic, or a polymer.

In some embodiments, the fluidic module defining the one or more fluidic path(s) is made of a material of the kind described herein.

Advantageously, the continuous synthesis of halogenated alkoxyethane in one or more fluidic path(s) of the kind described herein is more efficient than a corresponding synthesis performed in batch system according to conventional procedures. In that regard, fluid behaviour in a fluidic system of the kind described herein differs significantly from fluid behaviour in batch environments. While fluid dynamics in batch environments is mostly dominated by pressure and gravity, in the plate reactor of the invention surface tension, energy dissipation and fluidic resistance play a significant role in determining the fluid dynamics. In addition, mixing efficiency afforded by the tortuous nature of the one or more fluidic path(s) of the kind described herein is superior to that of conventional processes.

The internal cross-sectional area of the one or more fluidic path(s) may have any geometry. Examples of suitable geometries of the internal cross-sectional area include a circular geometry, a square geometry, a rectangular geometry, a triangular geometry, or other geometries known in the art.

The process of the invention comprises a step of introducing in the plate reactor reaction components comprising (i) a compound of general formula XClC═CF₂, (ii) a base, and (iii) a C₁₋₄ alkanol.

The compound of general formula XClC═CF₂ may be any compound of that formula in which X is —Cl or —F. In some embodiments, X is —Cl, in which case the compound of general formula XClC═CF₂ is Cl₂C═CF₂. In some embodiments, X is —F, in which case the compound of general formula XClC═CF₂ is FClC═CF₂.

The C₁₋₄ alkanol may be any C₁₋₄ alkanol that promotes addition reaction to the C═C bond of the compound of general formula XClC═CF₂, resulting in a C₁₋₄ alkoxy group bonded on the second carbon. In some embodiments, the C₁₋₄ alkanol is selected from methanol (CH₃OH), ethanol (CH₃CH₂OH), 1-propanol (CH₃CH₂CH₂OH), 2-propanol ((CH₃)₂CHOH), 1-butanol (CH₃CH₂CH₂CH₂OH), 2-butanol (CH₃CH₂CHOHCH₃), 2-methyl-1-propanol ((CH₃)₂CHCH₂OH), 2-methyl-2-propanol ((CH₃)₃COH), and a combination thereof. In some embodiments, the C₁₋₄ alkanol is methanol.

The base may be any base that can catalyse the addition reaction of the C₁₋₄ alkanol to the compound of general formula XClC═CF₂ under the conditions described herein. In other words, the base would be one that is strong enough to create corresponding alkoxy ions from the C₁₋₄ alkanol. When the C₁₋₄ alkanol is methanol, for example, the base would be one that is strong enough to create a methoxy ion.

In some embodiments, the base comprises an alkali metal base cation. For example, the base may be selected from the group consisting of an alkali metal (e.g. Li, Na and K), an alkali metal salt (e.g. carbonates, acetates and cyanides), an alkali metal hydroxide, an alkali metal alkoxide (e.g. methylate, ethylate, phenolate), and a combination thereof. For example, the base may be selected from sodium methoxide, and potassium methoxide. In some embodiments, the base is an alkali metal hydroxide of general formula M-OH, wherein M is an alkali metal selected from the group consisting of Li, Na and K. In some embodiments, the alkali metal hydroxide is NaOH or KOH. In some embodiments, the base is KOH.

Preferably, in some embodiments, the base comprises a nitrogen containing base. For example, an ammonium base. Examples of suitable such bases include tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide. In some embodiments, the base is a phosphonium base. For example, the base may be tetramethylphosphonium hydroxide.

It will be understood that the process of the invention can be advantageously performed with a single base, for example a single base of the kind described herein. This is opposed to, for instance, using a mixture of different bases providing a composite base catalyst system. Accordingly, in some embodiments the base used in the process of the invention is a single base. For instance, in some embodiments the base is one base selected from tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide, and tetramethylphosphonium hydroxide.

During formation of the halogenated alkoxyethane, salt intermediates may precipitate within the fluidic path(s). In those instances, precipitation of the intermediate salt could lead to undesired blockage of the fluidic path(s). The line(s) would have to be cleaned, leading to unwanted process interruptions. Examples of salt intermediates which may be expected to precipitate during the reaction include salts of an alkali metal (e.g. sodium salts, potassium salts), or halide salts (e.g. chloride, fluoride salts, such as Na fluoride or K fluoride). In those instances, a number of strategies may be adopted to minimise issues deriving from potential precipitation of salt intermediates.

For example, the base may be selected such that it forms a salt soluble in the alkanol during formation of the halogenated alkoxyethane. This advantageously minimises formation of insoluble precipitates along the fluidic path(s). As a result, the plate reactor can be operated without interrupting fluid flow through the line(s) for significantly longer times relative to conventional procedures. In addition, line cleaning can be less frequent and less onerous, resulting in significant cost savings. In this context, an intermediate salt would be considered “soluble” in the C₁₋₄ alkanol if the salt does not crystallise and precipitate under the reaction conditions. For example, an intermediate salt may be considered “soluble” in the C₁₋₄ alkanol if its solubility in the C₁₋₄ alkanol is at least 0.5 wt % under the reaction conditions. Suitable examples of bases that can form a salt that is soluble in the alkanol include a base comprising an ammonium or phosphonium base cation, such as one selected from tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide, and tetramethylphosphonium hydroxide.

For example, when the compound of general formula XClC═CF₂ is Cl₂C═CF₂, the base may be an alkylammonium hydroxide, an alkylammonium chloride, or an alkylphosphonium hydroxide. For example, the base may be selected from tetrabutylammonium hydroxide, benzyl(trimethyl)ammonium hydroxide, N-methyl-N,N,N-trioctylammonium chloride, tetraethylammonium hydroxide, tetramethylammonium hydroxide, and tetramethylphosphonium hydroxide. In those instances, formation and precipitation of salt intermediates can be minimised.

In some embodiments, the compound of general formula XClC═CF₂ is Cl₂C═CF₂ (1,1-dichloro-2,2-difluoroethylene) and the C₁₋₄ alkanol is methanol. In those instances, the process of the invention allows for the efficient and scalable production of halogenated alkoxyethane compounds such as methoxyflurane (Cl₂HC—CF₂OCH₃). This is particularly advantageous since methoxyflurane is the active ingredient of Penthrox®, which is an effective and rapid-onset short-term analgesic for the initial management of acute trauma pain and brief painful procedures such as wound dressing. Penthrox® is an analgesic used by medical practitioners, the defence forces, ambulance paramedics, sports clubs and surf lifesavers to administer emergency pain relief through inhaler devices known as “Green Whistles”.

Penthrox® has received Regulatory Approvals in a number of major jurisdictions around the world, and is expected to be ubiquitously available as disposable, single-use inhaler devices allowing patients (including children) to self-administer the drug under supervision. Current testing is being performed on advanced inhalers for the self-administration of Penthrox® to be marketed in addition to the Green Whistles. The test inhalers have been developed to be fully integrated pain release systems delivering about 3 ml of Penthrox® to patients in a quick and easy manner. The test inhaler comprises a lock out tab, a plunger that activates the inhaler, and a mouthpiece though which the user can inhale the active Penthrox® composition by normal breathing. Once the lock out tab is removed, the inhaler can be activated by pushing down the plunger. The inhaler would then be set to release the active ingredient through the mouthpiece by the user simply inhaling.

Penthrox® is aimed at becoming available worldwide in facilities that (i) can provide first-aid and emergency services (e.g. hospital emergency, ambulance services, life-saving clubs, etc.), (ii) necessitate mobile, agile, and point-of-care first-aid and emergency services (e.g. the military), and (iii) can market Penthrox® to the general public (e.g. pharmacies) as a mainstream analgesic of choice.

Certain process parameters are particularly advantageous for the production of pharmaceutical grade methoxyflurane using a plate reactor of the kind described herein.

For instance, it is particularly advantageous to effect formation of methoxyflurane at a temperature of from about −10° C. to about 25° C. Accordingly, in some embodiments the fluidic module(s) is/are at a temperature of from about −5° C. to about 15° C. In some embodiments, the fluidic module(s) is/are at a temperature of about 10° C.

In some embodiments, methoxyflurane is produced using a plate reactor comprising fluidic modules in which the one or more fluidic path(s) define successive chambers, each with a nozzle-like entrance and a narrowing exit. A chamber of said successive chambers may be nested with a next-succeeding chamber such that the narrowing exit of the one chamber forms the nozzle-like entrance of the next adjacent succeeding chamber. This configuration can be particularly advantageous in that it can provides a tortuous path for fluid flow, further contributing to the mixing of the reaction components.

In some embodiments, methoxyflurane is produced using a plate reactor comprising fluidic modules having characteristics described herein, for example characteristics of the modules depicted in any one of FIGS. 1-4.

In some embodiments, methoxyflurane is produced using a plate reactor comprising multiple fluidic modules providing for one or more fluidic path(s) having a total internal volume of at least 10 ml. For example, methoxyflurane may be produced using a plate reactor comprising multiple fluidic modules providing for one or more fluidic path(s) having a total internal volume of between about 10 ml and about 2 L. In some embodiments, the total internal volume is between about 20 ml and about 1 L, between about 20 ml and about 750 ml, between about 20 ml and about 500 ml, between about 20 ml and about 250 ml, between about 20 ml and about 100 ml, or between about 20 ml and about 50 ml.

Provided methoxyflurane forms, any base may be used. Examples of suitable bases for the synthesis of methoxyflurane include bases that comprise an alkali metal base cation. For example, the base may be selected from the group consisting of an alkali metal (e.g. Li, Na and K), an alkali metal salt (e.g. carbonates, acetates and cyanides), an alkali metal hydroxide, an alkali metal alkoxide (e.g. methylate, ethylate, phenolate), and a combination thereof. For example, the base may be selected from sodium methoxide, and potassium methoxide. In some embodiments, the base is an alkali metal hydroxide of general formula M-OH, wherein M is an alkali metal selected from the group consisting of Li, Na and K. In some embodiments, the alkali metal hydroxide is NaOH or KOH. In some embodiments, the base is KOH. In some embodiments, the base comprises an ammonium or phosphonium base cation. Examples of suitable such bases include tetrabutylammonium hydroxide, benzyl(trimethyl)ammonium hydroxide, N-methyl-N,N,N-trioctylammonium chloride (Aliquat 336), tetraethylammonium hydroxide, tetramethylammonium hydroxide, and tetramethylphosphonium hydroxide.

In some embodiments, methoxyflurane is produced by providing methanol and the base as a base/methanol solution. The solution may contain between about 1% (wt %) and about 10% (wt %) of the base relative to the total weight of the solution. For example, the solution may contain from about 2% (wt %) to about 5% (wt %) of the base relative to the total weight of the solution. In some embodiments, the base/methanol solution contains about 2.5% (wt %) of the base relative to the total weight of the solution. The base/methanol solution may be provided at a temperature between about −5° C. to about 10° C.

The base/methanol solution and Cl₂C═CF₂ may be mixed at any ratio conducive to formation of methoxyflurane. For example, the base/methanol solution and Cl₂C═CF₂ may be mixed according to a volume ratio of from 10:1 to 1:1. In some embodiments, the base/methanol solution and Cl₂C═CF₂ are mixed according to a volume ratio of 5:1. The appropriate volume ratio can be readily obtained by tuning the flow rate of each of the base/methanol solution and Cl₂C═CF₂ when they are mixed.

In some embodiments, the compound of general formula XClC═CF₂ is FClC═CF₂ and the C₁₋₄ alkanol is methanol. In those instances, the process of the invention affords efficient and scalable production of ClFHC—CF₂OCH₃ (2-chloro-1,1,2-trifluoroethylmethyl ether). The possibility to produce highly pure and high amounts of ClFHC—CF₂OCH₃ can be particularly advantageous, since that compound is a known precursor in the synthesis of the inhalant anaesthetic enflurane (2-chloro-1,1,2,-trifluoroethyl-difluoromethyl ether). In accordance to a reaction procedure postulated in Scheme 1 below, enflurane (b) can be synthesised by chlorinating ClFHC—CF₂OCH₃ in light (e.g. UV) to give 2-chloro-1,1,2-trifluoroethyldichloromethyl ether (a), followed by substitution of chlorine atoms by fluorine on the dichloromethyl group. The latter is achieved by using, for example, hydrogen fluoride in the presence of antimony(III) chloride, or antimony(III) fluoride with antimony(V) chloride.

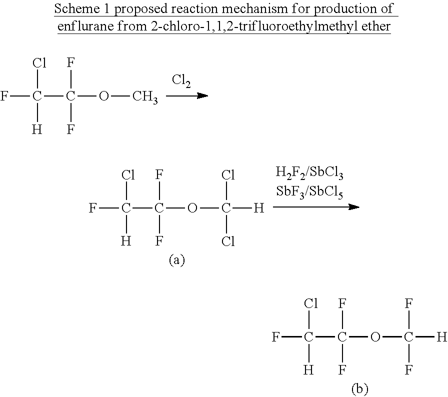

In the method of the invention, the base may be used in any amount conducive to the formation of the halogenated alkoxyethane. In a typical procedure, the base is used in a catalytic amount relative to the compound of general formula XClC═CF₂. By being used in a “catalytic amount”, the base is used in a substoichiometric amount relative to the compound of general formula XClC═CF₂. It will be understood that in the context of the plate reactor of the invention, the base is fed continuously into the plate reactor in a catalytic amount relative to the compound of general formula XClC═CF₂. In some embodiments, the base to XClC═CF₂ compound molar ratio is any decimal fraction of 1. For example, the base to XClC═CF₂ compound molar ratio may be about 0.1:1, about 0.15:1, about 0.2:1, about 0.25:1, about 0.3:1, about 0.4:1, about 0.5:1, about 0.6:1, about 0.7:1, about 0.8:1, or about 0.9:1.

In some embodiments, the base is used in solution with the C₁₋₄ alkanol. In those instances, the base/alkanol solution may contain the base in an amount between 1% and 30% by weight relative to the total weight of base and C₁₋₄ alkanol. For example, the base may be used in an amount of between about 1% and about 15% by weight, between about 1% and about 10% by weight, or between about 1% and about 5% by weight, relative to the total weight of base and C₁₋₄ alkanol. In some embodiments, the base is used in an amount of about 2.5% by weight relative to the total weight of base and C₁₋₄ alkanol. In some embodiments, the base is used in an amount of about 5% by weight relative to the total weight of base and C₁₋₄ alkanol. In some embodiments, the base is used in an amount of about 2.5% by weight relative to the total weight of base and C₁₋₄ alkanol.

It will be understood that the process of the invention can be advantageously implemented without additional reaction components to (i) the compound of general formula XClC═CF₂, where X is —Cl or —F, (ii) the base, and (iii) the C₁₋₄ alkanol, (i)-(iii) being reaction components of the kind described herein.

For example, in the context of the invention the C₁₋₄ alkanol may be said to act simultaneously as a reagent and solvent, such that the reaction proceeds with no need for the use of additional solvents other than the C₁₋₄ alkanol. For instance, one would understand that the process of the invention can be advantageously carried out without the need to use solvents which may conventionally be used in reactions involving chlorofluoro-olefins (e.g. N,N-dimethylformamide (DMF), N-methylpyrrolidone (NMP), dimethyl sulfoxide (DMSO), sulfolane, diethylene glycol dimethyl ether (DG)), or tetraethylene glycol dimethyl ether (TG)).

Accordingly, the invention may also be said to provide a process for continuous preparation of halogenated alkoxyethane of general formula XClHC—CF₂OR, where X is —Cl or —F and OR is C₁₋₄ alkoxy, the process comprising a step of introducing in a plate reactor reaction components consisting of (i) a compound of general formula XClC═CF₂, where X is —Cl or —F, (ii) a base, and (iii) a C₁₋₄ alkanol, wherein
a) the plate reactor comprises a fluidic module defining one or more fluidic path(s) through which the reaction components flow as a reaction mixture, and
b) the halogenated alkoxyethane is formed at least upon the reaction components mixing, with the so formed halogenated alkoxyethane flowing out of the plate reactor in a reactor effluent.

For instance, when the process of the invention is used to prepare methoxyflurane, the invention may be said to provide a process for continuous preparation of 2,2-dichloro-1,1-difluoro-1-methoxyethane (methoxyflurane), the process comprising a step of introducing in a plate reactor reaction components consisting of (i) 1,1-dichloro-2,2-difluoroethene (Cl₂C═CF₂), (ii) a base, and (iii) methanol, wherein
a) the plate reactor comprises a fluidic module defining one or more fluidic path(s) through which the reaction components flow as a reaction mixture, and
b) the methoxyflurane is formed at least upon the reaction components mixing, with the so formed methoxyflurane flowing out of the plate reactor in a reactor effluent.

In addition, when the process of the invention is used to prepare ClFHC—CF₂OCH₃, the invention may be said to provide a process for continuous preparation of ClFHC—CF₂OCH₃, the process comprising a step of introducing in a plate reactor reaction components consisting of (i) FClC═CF₂, (ii) a base, and (iii) methanol, wherein
a) the plate reactor comprises a fluidic module defining one or more fluidic path(s) through which the reaction components flow as a reaction mixture, and
b) the ClFHC—CF₂OCH₃ is formed at least upon the reaction components mixing, with the so formed ClFHC—CF₂OCH₃ flowing out of the plate reactor in a reactor effluent.

In the process of the invention, the reaction components flow through the one or more fluidic path(s) as a reaction mixture. Typically, each reaction component will be provided as a discrete component, and the components mixed to form the reaction mixture. Mixing of the components may be achieved according to any sequence or means suitable to ensure that the components flow through the one or more fluidic path(s) as a reaction mixture. For example, each component may be provided in corresponding separate reservoirs, from which they are extracted (e.g. pumped) and mixed with the other components to form the reaction mixture. Said mixing may be performed according to any suitable mixing sequence.

In some embodiments, the reaction components are mixed upstream of the one or more fluidic path(s). In those instances, the fluid that is introduced into the one or more fluidic path(s) is the reaction mixture.

In some preferred embodiments, the reaction components are introduced (e.g. pumped) into discrete fluidic paths of a fluidic module, for example through corresponding dedicated inlets, and made to mix by designing the fluidic paths so that they merge.

In some embodiments, the base and the C₁₋₄ alkanol are provided as a solution of the kind described herein in a first reservoir, and the XClC═CF₂ compound in a second reservoir. In those instances, the reaction mixture is therefore obtained by mixing (i) the solution of the base and the C₁₋₄ alkanol extracted from the first reservoir with (ii) the compound of general formula XClC═CF₂ extracted from the second reservoir. Said mixing may be effected upstream of the one or more fluidic path(s), and the mixture subsequently made to flow (e.g. pumped) through the one or more fluidic path(s). Alternatively, in some preferred arrangements said mixing may be effected along the one or more fluidic path(s), for example by adopting fluidic modules defining merging fluidic path(s).

Arrangements in which mixing of the XClC═CF₂ compound, the base, and the C₁₋₄ alkanol (in any combination, e.g. mixing of the XClC═CF₂ compound with a solution of the base and the C₁₋₄ alkanol, or the mixing of the XClC═CF₂ compound, the base, and the C₁₋₄ alkanol as discrete compounds) is achieved within a fluidic module (e.g. by having fluidic paths that merge) are particularly advantageous for the production of methoxyflurane.

In those instances where the base, the alkanol, and the XClC═CF₂ compound are mixed upstream of the one or more fluidic path(s), the base, the alkanol, and the XClC═CF₂ compound may be mixed to form the reaction mixture by any means known to the skilled person.

In some instances, the base, the alkanol (or a base/alkanol solution) and the XClC═CF₂ compound are mixed by flowing them through lines that interject to form a single fluidic line, for example in a T- or Y-configuration. In those cases, the resulting single fluidic line may be the feed of the one or more fluidic path(s) of the plate reactor.

In yet further configurations, the base, the alkanol (or a base/alkanol solution) and the XClC═CF₂ compound are mixed in a mixing unit located upstream of the one or more fluidic path(s). This can advantageously ensure a high degree of mixing between all reaction components before they enter into the fluidic path(s) as a reaction mixture. As a result, fast formation of highly pure alogenated alkoxyethane can be achieved, even in the absence of static mixers within the fluidic path(s).

The mixing unit may or may not be an integral component of the plate reactor. The mixing unit may be an active mixing unit, in which mixing is achieved by providing external energy. Examples of such units suitable for use in the process of the invention include units that impart time-pulsing flow owing to a periodical change of pumping energy or electrical fields, acoustic fluid shaking, ultrasound, electrowetting-based droplet shaking, micro-stirrers, and the like. In alternative configurations, the mixing unit may be a passive mixing unit, in which mixing is achieved by combining the base/alkanol solution line and the XClC═CF₂ compound line into one single line. Examples of such units suitable for use in the process of the invention include Y- and T-type flow junctions, multi-laminating mixers, split-and-recombine mixers, chaotic mixers, jet colliding mixers, recirculation flow-mixers, and the like. Typical design for passive mixing units include T- and Y-flow configurations, interdigital- and bifurcation flow distribution structures, focusing structures for flow compression, repeated flow division- and recombination structures, flow obstacles within the line, meander-like or zig-zag channels, multi-hole plates, tiny nozzles, and the like.

In some embodiments, the one or more fluidic path(s) comprise an inline static mixer. This is particularly advantageous to complement diffusion-driven intermixing of the components as they flow through the one or more fluidic path(s) (which can be a major driver of mixing in fluidic path(s) of small internal cross-sectional area). A static mixer within the fluidic path(s) can therefore be implemented to induce multi-lamellation of the flowing fluid or the formation of vortices within the volume of the flowing fluid, thereby increasing mixing efficiency.

Examples of suitable static mixers include baffles, helical mixers, spinning disks, and spinning tubes. As the skilled person will appreciate, the static mixer may be made of any material that is chemically inert to the reaction components, the halogenated alkoxyethane, and any reaction by-product and/or intermediate. Examples of suitable materials in that regard include polyethylene, polypropylene, polyvinyl chloride, a fluorocarbon (e.g. Teflon, polytetrafluoroethylene, polyvinylidene fluoride, fluorinated ethylene propylene, ethylene chlorotrifluoroethylene, polyvinylidene difluoride, a perfluoroalkoxy alkane, etc.), polyether ether ketone, polyethylene, fiberglass-reinforced plastic, silicon carbide, silica, Ni-based alloy, and No-Mo-based alloy. The skilled person would be readily capable to identify other materials suitable for use in the static mixer.

Examples of a suitable configuration of static mixers are provided by baffles (5) and curved baffles (9) in the embodiment fluidic modules of FIGS. 1-4.

While the above discussion is made in the context of materials used to make the internal walls of the one or more fluidic path(s), it will be understood that similar considerations apply to the material used to make (or internally line/coat) any element (or part thereof) of the system/apparatus used to perform the process and that is expected to come into contact with any one of the reaction components, product, intermediate, by-product(s), and/or mixture thereof. That is, it will be understood that any element (or part thereof) of the system/apparatus used to perform the process that is expected to come into contact with any one of the reaction components, product, intermediate, by-product(s), and/or mixture thereof would have to be made of a material that is chemically inert to said reaction component, product, intermediate, by-product(s) (which may include strong acids such as HCl or HF), and/or mixture thereof. Accordingly, any such element(s) may be made (or lined with, as appropriate) by a material of the kind described herein.

For example, any reservoir that is part of the system/apparatus used to perform the process may be made of (or internally lined with) a material that is chemically inert to the chemical component or mixture the reservoir is intended to store. Similarly, relevant components of pumps that may be used to pump a reaction component, product, intermediate, by-product(s), and/or any mixture thereof may be made of a material that is chemically inert to said reaction component, product, intermediate, by-product(s), and/or mixture thereof. Also, relevant components of mixing units of the kind described herein which may come into contact with a reaction component, product, intermediate, by-product(s), and/or any mixture thereof may be made of a material that is chemically inert to said reaction component, product, by-product(s), and/or mixture thereof. Examples of suitable materials in that regard include polyethylene, polypropylene, polyvinyl chloride, a fluorocarbon (e.g. Teflon, polytetrafluoroethylene, polyvinylidene fluoride, fluorinated ethylene propylene, ethylene chlorotrifluoroethylene, polyvinylidene difluoride, a perfluoroalkoxy alkane, etc.), polyether ether ketone, polyethylene, fiberglass-reinforced plastic, Ni-based alloy, and No-Mo-based alloy. The skilled person would be readily capable to identify other materials suitable for use in any of the components of the reactor to ensure safe handling of all mixtures and compounds involved in the invention.

In the process of the invention, the relative amount of the reaction components in the reaction mixture can be modulated by tuning the flow rate of each component when it is mixed with the others.

For example, when the base and alkanol are provided as a base/alkanol solution, the relative amount of the reaction components in the reaction mixture can be modulated by tuning the flow rate of the base/alkanol solution relative to that of the XClC═CF₂ compound. The ratio between the flow rate of the base/alkanol solution relative to that of the compound of general formula XClC═CF₂ may be any ratio that is conducive to the formation of the halogenated alkoxyethane. For example, the reaction mixture may be obtained by combining (i) a solution of the C₁₋₄ alkanol and the base with (ii) the compound of general formula XClC═CF₂ according to a flow rate ratio from 1:1 to 10:1. In some embodiments, said flow rate ratio is between 1:1 to 6:1, from 2:1 to 6:1, from 3:1 to 6:1, or from 4:1 to 5:1.

In that context, each of the base/alkanol solution line and the XClC═CF₂ compound line may be operated at a flow rate that is conducive to the formation of the halogenated alkoxyethane upon mixing of the base/alkanol solution with the XClC═CF₂ compound. In one embodiment, the flow rate of each individual line is at least 1 ml/min. For example, the flow rate of each individual line may be at least about 5 ml/min, at least about 25 ml/min, at least about 50 ml/min, at least about 100 ml/min, at least about 200 ml/min, at least about 500 ml/min, at least about 1,000 ml/min, at least about 1,500 ml/min, at least about 2,000 ml/min, at least about 4,000 ml/min, or at least about 8,000 ml/min. In some embodiments, the flow rate of each individual line is about 250 ml/min.

In some embodiments, the base/alkanol solution is pumped or otherwise supplied into the mixer unit or the one or more fluidic path(s) at a flow rate greater than 5 ml/min but less than 8,000 ml/min, and the XClC═CF₂ compound is pumped or otherwise supplied into the mixer unit or the one or more fluidic path(s) at a flow rate greater than 5 ml/min but less than 8,000 ml/min. In one embodiment, the base/alkanol solution is pumped or otherwise supplied into the mixer unit or the one or more fluidic path(s) at a flow rate greater than or equal to 50 ml/min but less than or equal to 500 ml/min, and the XClC═CF₂ compound is pumped or otherwise supplied into the mixer unit or the one or more fluidic path(s) at a flow rate greater than or equal to 50 ml/min but less than or equal to 500 ml/min. In one embodiment, the base/alkanol solution is pumped or otherwise supplied into the mixer unit or the one or more fluidic path(s) at a flow rate of about 250 ml/min, and the XClC═CF₂ compound is pumped or otherwise supplied into the mixer unit or the one or more fluidic path(s) at a flow rate of about 50 ml/min.

In the process of the invention, the halogenated alkoxyethane flows out of the plate reactor in a reactor effluent. This may be achieved by any means known to the skilled person. When the plate reactor comprises two or more fluidic paths, the lines would typically converge to form a single outlet from which the effluent exits the reactor. The effluent may exit the reactor at a flow rate that depends on the operational parameters of the reactor. For example, the reactor effluent containing the halogenated alkoxyethane may exit the reactor at a flow rate of at least 5 ml/min. In some embodiments, the reactor effluent containing the halogenated alkoxyethane exits the reactor at a flow rate of at least 10 ml/min, at least 25 ml/min, at least 50 ml/min, at least 100 ml/min, at least 250 ml/min, at least 500 ml/min, at least 750 ml/min, at least 1 L/min, at least 1.5 L/min, at least 2 L/min, at least 4 L/min, or at least 8 L/min.

The effluent may contain an amount of halogenated alkoxyethane that is dependent on the operational parameters of the reactor. In some embodiments, the reactor effluent contains at least 70% by volume, at least 80% by volume, at least 90% by volume, or at least 95% by volume of the halogenated alkoxyethane. Advantageously, the process of the invention affords higher conversion yields than conventional procedures. Accordingly, in some embodiments the reactor effluent contains at least 90% by volume of the halogenated alkoxyethane. In other words, the reactor effluent contains the halogenated alkoxyethane at a purity of 70% or above, for example 80% or above, 90% or above, or 95% or above.

In some embodiments, the process also comprises a step of mixing the reactor effluent with a polar liquid. For example, the process may comprise a step of mixing the reactor effluent with water. This may provide a biphasic mixture that can be used in the context of the purification procedure described herein. The polar liquid (e.g. water) may be mixed with the reactor effluent by any of the mixing procedures described herein. For example, one or more lines carrying the polar liquid (e.g. water) from a reservoir may be made to interject the reactor effluent line, and the polar liquid made to flow (e.g. pumped) from a dedicated reservoir. Alternatively, the polar liquid (e.g. water) may be mixed with the reactor effluent by way of a mixing unit of the kind described herein.

The polar liquid (e.g. water) may be provided according to any flow rate that is suitable to obtain a biphasic mixture with the reactor effluent. Typically, the polar liquid (e.g. water) may be pumped at room temperature.

The reactor effluent may also contain additional compounds present in the effluent as impurities. Depending on the reactor conditions and/or the nature of the reaction components, said impurities may comprise one or more reaction by-product(s) and/or one or more unreacted reaction components. The nature of the impurities depends on the reaction conditions and/or the nature of the reaction components. For example, when the process of the invention is performed to produce methoxyflurane, the impurities may comprise one or more of methanol, dichloro-difluoroethylene (DCDFE), 2,2-dichloro-1,1,1-trifluoroethane, chloroform, ethers (for example vinyl ethers such as methoxyethene (ME), 1,1-dichloro-2-fluoro-2-methoxyethene, halomar (2-chloro-1,1,2-trifluoroethyl methyl ether)), orthoesters (OE) such as 2,2-dichloro-1,1,1-trimethoxyethane, methyl dichloroacetate (MDA), chloroform, and HF. In one such embodiments, the impurities comprise 1,1-dichloro-2-fluoro-2-methoxyethene.

Accordingly, in some embodiments the process is one for purifying the halogenated alkoxyethane from impurities comprising one or more of methanol, 2,2-dichloro-1,1,1-trifluoroethane, methyl dichloroacetate, 1,1-dichloro-2,2-difluoroethylene, chloroform, hydrogen fluoride and methoxyethene (ME), orthoesters (OE) such as 2,2-dichloro-1,1,1-trimethoxyethane, and methyl dichloroacetate (MDA).

Depending on the reactor conditions and/or the nature of the reaction components, said impurities may also be present in an amount that can range from less than 5% up to about 30% by volume of the effluent. Advantageously, the process of the invention can ensure that the halogenated alkoxyethane can be produced at a significantly higher purity (i.e. above 90% by volume of effluent) relative to conventional synthesis procedures. In some embodiments, the reactor effluent contains less than 5% impurities by volume.

If necessary, as part of the process of the invention, the halogenated alkoxyethane exiting the plate reactor in the effluent may be subject to purification.

Accordingly, in some embodiments the process of the invention further comprises a purification procedure that comprises the steps of:
a) adding one of an amine and an acid to the reactor effluent or an organic phase separated from the reactor effluent,
b) adding a polar liquid to the mixture obtained in step a) to induce phase separation and formation of a polar phase and a separate organic phase, the organic phase containing the halogenated alkoxyethane,
c) adding the other of the amine and the acid not used in step a) to the organic phase obtained in step b), to thereby purify the halogenated alkoxyethane.

In this context, by the procedure being a “purification” procedure is meant that said procedure affords removal of impurities from the reactor effluent or an organic phase separated from the reactor effluent, for example impurities of the kind described herein, resulting in a mixture having less amount of impurities relative to the reactor effluent or an organic phase separated from the reactor effluent.

In some embodiments, the purification procedure comprises a step d) of isolating the purified halogenated alkoxyethane. In step d), the purified halogenated alkoxyethane may be isolated by any suitable means known to a skilled person that would result in halogenated alkoxyethane with purity of at least 95%, for example at least 99%, such as about 99.9%. Accordingly, the present invention may also be said to provide a halogenated alkoxyethane of general formula XClHC—CF₂OR, where X is —Cl or —F and OR is C₁₋₄ alkoxy, obtained in accordance with the process described herein, the halogenated alkoxyethane having purity of at least 99%.

Accordingly, in some embodiments the process of the invention further comprises a purification procedure that comprises the steps of:
a) adding one of an amine and an acid to the reactor effluent or an organic phase separated from the reactor effluent,
b) adding a polar liquid to the mixture obtained in step a) to induce phase separation and formation of a polar phase and a separate organic phase, the organic phase containing the halogenated alkoxyethane,
c) adding the other of the amine and the acid not used in step a) to the organic phase obtained in step b), and
d) isolating the purified halogenated alkoxyethane.

In some embodiments, the purification procedure is performed directly on the reactor effluent.

In some embodiments, the reactor effluent undergoes further processing before adding the amine or the acid. For example, the reactor effluent may first undergo a phase separation procedure. Said procedure may involve the addition of a polar liquid (e.g. water) to the reactor effluent to form a biphasic mixture made of a polar phase and a separate organic phase comprising the halogenated alkoxyethane. In those instances, the organic phase would then be separated from the polar phase, which can be discarded, before further processing. The phase separation can be effected as a batch or continuous (e.g. in-line) phase separation.

Accordingly, in some embodiments the process of the invention further comprises adding a polar liquid to the reactor effluent to induce phase separation and formation of a polar phase and a separate organic phase, and separating said organic phase from the polar phase. Said organic phase is the organic phase separated from the reactor effluent mentioned in step a).

In the contest of the purification procedure, separation of a polar phase from a separate organic phase in a biphasic mixture may be effected according to any means known to the skilled person. For example, said separation may be effected by way of a gravity separator (e.g. a phase separation flask, tank, or a separating funnel), a super-hydrophobic mesh, a super-oleophobic mesh, and the like. A skilled person would be capable to identify suitable means and procedures for the effective separation of the phases of a biphasic mixture.

As used herein, a “polar liquid” is a liquid substance that can be added to a mixture comprising a halogenated alkoxyethane of the kind described herein, resulting in the formation of a biphasic mixture comprising a polar phase and a separate organic phase containing the halogenated alkoxyethane. An example of a suitable polar liquid in that regard is water.

The purification procedure comprises a step a) of adding one of an amine and an acid to the reactor effluent or an organic phase separated from the reactor effluent. In this step, either an amine or an acid is added to the reactor effluent or an organic phase separated from the reactor effluent. Accordingly, in some embodiments the purification procedure comprises adding an amine to the reactor effluent or an organic phase separated from the reactor effluent. In some embodiments, the purification procedure comprises adding an acid to the reactor effluent or an organic phase separated from the reactor effluent. The amine or the acid may be an amine or an acid of the kind described herein.

In some embodiments, step a) of the purification procedure comprises adding an amine to the reactor effluent or an organic phase separated from the reactor effluent.

The amine may be a primary or a secondary amine.

Without wanting to be limited by theory, it is believed that an amine of the kind described herein can react with impurities present in the reactor effluent or an organic phase separated from the reactor effluent through N-alkylation and/or amidation routes. This advantageously converts the impurities into compounds that are more amenable to removal in the isolation step than the starting impurities.

For example, a synthetic procedure for producing methoxyflurane of the kind described herein can lead to the formation of 1,1-dichloro-2-fluoro-2-methoxyethene (vinyl ether) and/or methyl dichloroacetate impurities. In those instances, 1,1-dichloro-2-fluoro-2-methoxyethene (vinyl ether) can react with primary and/or secondary amines through N-methylation, providing 2,2-dichloroacetyl fluoride. Both 2,2-dichloroacetyl fluoride and methyl dichloroacetate may react further with primary and/or secondary amines through amidation routes to produce corresponding dichloroacetamides. The resulting dichloroacetamides are more amenable to removal in the isolation step. A schematic of those reactions is shown in Scheme 2.

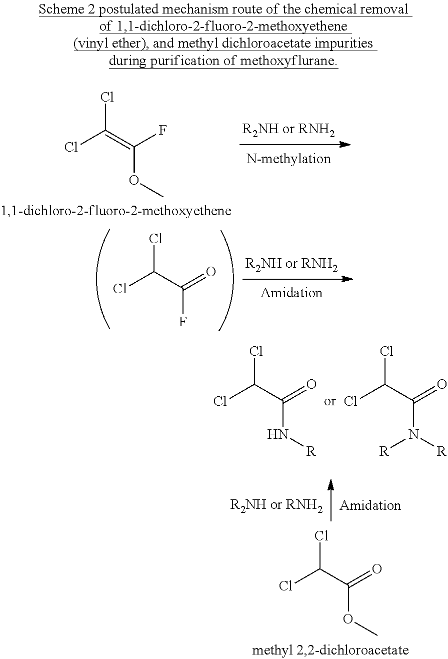

Examples of amines suitable for use in the purification procedure include ethylenediamine (1,2-diamnoethane), 1,3-diaminopropane, diethylenetriamine, di-n-propylamine, n-butylamine, ethanolamine, pyrrolidine, 2-aminobutane, and a mixture thereof. In some embodiments, the amine is selected from ethylenediamine, 1,3-diaminopropane, diethylenetriamine, and a mixture thereof.

In some embodiments, step a) of the purification procedure comprises adding an acid to the reactor effluent or an organic phase separated from the reactor effluent.

Examples of suitable acids include citric acid, hydrochloric acid, sulfuric acid, sulphurous acid, methanesulfonic acid, trifluoromethanesulfonic acid, phosphoric acid, acetic acid, trifluoroacetic acid, nitric acid, nitrous acid, hypochlorous acid, chlorous acid, chloric acid, perchloric acid, and a combination thereof. In one embodiment, the acid is methanesulfonic acid (MSA).

The acid may be added in any form that would be suitable to promote effective reaction with impurities present in the reactor effluent or an organic phase separated from the reactor effluent. For example, the acid may be in the form of an acid solution, such as an aqueous acid solution.

In some embodiments, the acid is at least a 10%, at least a 20%, at least at 30%, or at least a 40% acid solution.

In step a) of the purification procedure, the amine or the acid may be added to the reactor effluent or an organic phase separated from the reactor effluent according to any effective amount that is fit for the intended purpose. In some embodiments, the amine or the acid are added to the reactor effluent or an organic phase separated from the reactor effluent according to a volume ratio from about 0.05:1 to about 2:1 (amine or acid:reactor effluent or an organic phase separated from the reactor effluent). In some embodiments, the amine or the acid are added to the reactor effluent or an organic phase separated from the reactor effluent according to a volume ratio of about 0.1:1, about 0.25:1, about 0.5:1, about 1:1, or about 2:1 (amine or acid:reactor effluent or an organic phase separated from the reactor effluent).

Step a) of the purification procedure may be performed in any manner that is effective to promote reaction between one or more impurities and the amine or the acid. For example, addition of the amine or the acid may be performed as a batch procedure or as a continuous procedure.

Once the amine or the acid is added to the reactor effluent or an organic phase separated from the reactor effluent in step a) of the purification procedure, the resulting mixture can be let to react for any duration of time conducive to effective reaction between one or more impurities and the amine or the acid. For example, the mixture obtained in step a) of the purification procedure may be let to react for at least about 1 minute. In some embodiments, the mixture obtained in step a) of the purification procedure is let to react for at least about 5 minutes, at least about 15 minutes, at least about 30 minutes, at least about 60 minutes, or at least about 2 hours. During reaction, the mixture may be kept under constant stirring.

Addition of the amine or the acid to the reactor effluent or an organic phase separated from the reactor effluent in step a) of the purification procedure may be performed at any temperature conducive to effective reaction between one or more impurities and the amine or the acid. For example, the amine or the acid may be added to the reactor effluent or an organic phase separated from the reactor effluent at a temperature of from about 10° C. to about 120° C. High addition temperatures (e.g. up to 120° C.) may facilitate separation of more volatile impurities. In some embodiments, the amine or the acid is added to the reactor effluent or an organic phase separated from the reactor effluent at a temperature of from about 10° C. to about 50° C. In some embodiments, the amine or the acid in step a) of the purification procedure is added to the reaction mixture at room temperature. The resulting mixture may be kept at a temperature that is conducive to effective reaction between one or more impurities and the amine or the acid. For example, the resulting mixture may be kept at a temperature of from about 10° C. to about 50° C. In some instances, reaction between impurities and the amine or the acid can be exothermic, in which case following addition of the amine or the acids the temperature of the resulting mixture may be observed to increase gradually as the amine or the acid are added.

The purification procedure also comprises a step b) of adding a polar liquid to the mixture obtained in step a) of the purification procedure. This results in formation a biphasic mixture made of a polar phase and a separate organic phase, in which the separate organic phase contains the halogenated alkoxyethane.

The polar liquid used in step b) of the purification procedure may be a polar liquid of the kind described herein. For example, the polar liquid used in step b) of the purification procedure may be water. In those instances, the polar phase in step b) would be an aqueous phase.

In step b) of the purification procedure, the polar liquid may be added to the mixture obtained in step a) of the purification procedure in any amount suitable to induce the required phase separation and formation of a polar phase and a separated organic phase. For example, the polar liquid may be added to the mixture obtained in step a) of the purification procedure according to a volume ratio from about 0.5:1 to about 2:1 (polar liquid:mixture). In some embodiments, the polar liquid is added to the mixture obtained in step a) of the purification procedure according to a volume ratio of about 0.5:1, about 1:1, about 1.5:1, or about 2:1 (polar liquid:mixture).

Once the polar liquid is added in step b) to the mixture obtained in step a) of the purification procedure, the resulting biphasic mixture may be maintained under stirring for any duration of time conducive to the dissolution of polar impurities present in the starting mixture into the polar phase. For example, the resulting biphasic mixture may be kept under constant stirring for at least about 5 minutes, at least about 15 minutes, at least about 30 minutes, or at least about 60 minutes.

In some embodiments, step b) of the purification procedure is followed by a step of separating the organic phase obtained in step b) from the polar phase before further processing. Separation may be effected according to any procedure known to a skilled person which would be fit for the intended purpose. For example, separation may be effected by means of the kind described herein. In those instances, the separated polar phase is discarded.

The purification procedure also comprises a step c) of adding the other of the amine and the acid not used in step a) to the organic phase obtained in step b).

By the expression “the other of the amine and the acid not used in step a)” is meant that if the amine is used in step a) of the purification procedure, then the acid is used in step c) of the purification procedure. Vice versa, if the acid is used in step a), then the amine is used in step c).

In some embodiments, the purification procedure comprises adding an amine to the reactor effluent or an organic phase separated from the reactor effluent, and a subsequent addition of an acid to the resulting mixture. The amine or the acid may be an amine or an acid of the kind described herein.

In some embodiments, the purification procedure comprises adding an acid to the reactor effluent or an organic phase separated from the reactor effluent, and a subsequent addition of an amine to the resulting mixture. The amine or the acid may be an amine or an acid of the kind described herein.

Accordingly, in some embodiments the purification procedure comprises the steps of:
i. adding an amine to the reactor effluent or an organic phase separated from the reactor effluent,
ii. adding a polar liquid to the mixture obtained in step i) to induce phase separation and formation of a polar phase and a separate organic phase, the organic phase containing the halogenated alkoxyethane,
iii. adding an acid to the organic phase obtained in step ii).

In some alternative embodiments, the purification procedure comprises the steps of:
i. adding an acid to the reactor effluent or an organic phase separated from the reactor effluent,
ii. adding a polar liquid to the mixture obtained in step i) to induce phase separation and formation of a polar phase and a separate organic phase, the organic phase containing the halogenated alkoxyethane,
iii. adding an amine to the organic phase obtained in step ii).

In the embodiments described in the previous four paragraphs, it will be understood that all the compounds (e.g. the amine, the acid, and the polar liquid) would be compounds of the kind described herein, and that any procedural condition would be a procedural condition of the kind described herein.

As a skilled person would appreciate, the addition of the amine or the acid to the organic phase obtained in step b) may require first separating said organic phase from the polar phase obtained in step b). For instance, when the amine or the acid used in step c) may react dangerously with the polar phase obtained in step b), the organic phase and said polar phase would have to be first separated. Phase separation may be achieved in accordance to any procedure of the kind described herein.

In step c) of the purification procedure, adding the other of the amine and the acid not used in step a) of the purification procedure to the organic phase obtained in step b) of the purification procedure is advantageous to convert impurities that could not be converted in step a), and/or eliminate undesired by-product impurities generated by reactions promoted in step a).

For example, when step a) of the purification procedure comprises adding an acid to the reactor effluent or an organic phase separated from the reactor effluent, ethane impurities (if any) may convert to the corresponding chloroacetates, which may impact the isolation of the purified halogenated alkoxyethane resulting in formation of further acidic by-product impurities. In turn, this may lead to contamination of the final product by chloroacetates. For instance, under acidic conditions the by-product 2,2-dichloro-1,1,1-timethoxyethane may be converted to methyl dichloroacetate as summarised in Scheme 3 below.

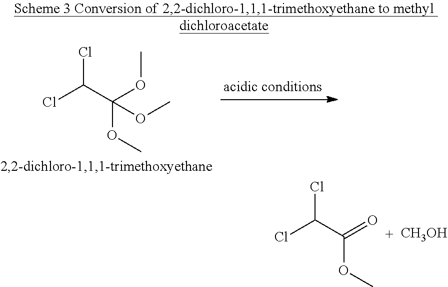

In those instances, the amine subsequently added in step c) of the purification procedure can react with the chloroacetates through amidation routes to produce corresponding dichloroacetamides, which are more amenable to removal in the isolation step.

In step c) of the purification procedure, the amine or the acid may be added to the organic phase obtained in step b) according to any effective amount that is fit for the intended purpose. In some embodiments, the amine or the acid are added to the organic phase obtained in step b) according to a volume ratio from about 0.05:1 to about 2:1 (amine or acid:organic phase). In some embodiments, the amine or the acid are added to the organic phase obtained in step b) according to a volume ratio of about 0.1:1, about 0.25:1, about 0.5:1, about 1:1, or about 2:1 (amine or acid:organic phase).

Step c) of the purification procedure may be performed in any manner that is effective to promote reaction between one or more impurities and the amine or the acid. For example, addition of the amine or the acid to the organic phase obtained in step b) of the purification procedure may be performed as a batch procedure or as a continuous procedure.

In step c) of the purification procedure, once the amine or the acid is added to the organic phase of step b), the resulting mixture can be let to react for any duration of time conducive to effective reaction between one or more impurities and the amine or the acid. For example, the mixture obtained in step c) of the purification procedure may be let to react for at least about 1 minute. In some embodiments, the mixture obtained in step c) of the purification procedure is let to react for at least about 5 minutes, at least about 15 minutes, at least about 30 minutes, at least about 60 minutes, or at least about 2 hours. During reaction, the mixture may be kept under constant stirring.

Addition of the amine or the acid in step c) of the purification procedure may be performed at any temperature conducive to effective reaction between one or more impurities and the amine or the acid. For example, in step c) of the purification procedure the amine or the acid may be added at a temperature of from about 10° C. to about 120° C. High addition temperatures (e.g. up to 120° C.) may facilitate separation of more volatile impurities. In some embodiments, the amine or the acid is added in step c) at a temperature of from about 10° C. to about 50° C. In some embodiments, the amine or the acid in step c) of the purification procedure are added at room temperature. The resulting mixture may be kept at a temperature that is conducive to effective reaction between one or more impurities and the amine or the acid. For example, the resulting mixture may be kept at a temperature of from about 10° C. to about 50° C.

Advantageously, the amine or the acid used in the purification procedure can react particularly effectively with impurities while remaining inert towards the halogenated alkoxyethane.

For example, in a purification procedure to obtain pharmaceutical grade methoxyflurane, an amine of the kind described herein is particularly effective to react selectively with low component impurity (e.g. methyl dichloroactetate) while retaining methoxyflurane. This has been found to be particularly advantageous for purifying methoxyflurane above 99% purity, for example at about 99.9% purity.

In a particularly advantageous purification procedure for methoxyflurane, step a) of the purification procedure comprises adding an acid to the reactor effluent or an organic phase separated from the reactor effluent, and step c) of the purification procedure comprises adding an amine to the organic phase obtained in step b). For instance, step a) of the purification procedure for methoxyflurane may comprise adding methane sulfonic acid to the reactor effluent or an organic phase separated from the reactor effluent, and step c) of the purification procedure may comprises adding ethanolamine to the organic phase obtained in step b). Accordingly, in some embodiments the process is one for the production of methoxyflurane, and includes a purification procedure comprising adding and acid (e.g. methane sulfonic acid) to the reactor effluent or an organic phase separated from the reactor effluent, and a subsequent addition of an amine (e.g. ethanolamine) to a resulting mixture.

Since the amine and the acid remain inert towards the halogenated alkoxyethane, the purification procedure can be performed using excess of amine and acid relative to the amount of impurities present in the relevant mixtures. Accordingly, any differences in the level of impurities depending on the specific synthesis procedure used to produce the halogenated alkoxyethane can be advantageously accommodated.

In short, the purification procedure in accordance with certain embodiments of the invention can facilitate removal of impurities from a mixture comprising the halogenated alkoxyethane irrespective of the amount of impurities present in the mixture. This is particularly advantageous when the synthesis of halogenated alkoxyethane is limited by low conversion yields. In those instances, the purification procedure of the invention can greatly assist to provide pharmaceutical grade halogenated alkoxyethane.

In some embodiments, the purification procedure comprises a step of adding a polar liquid to the mixture obtained in step c) of the purification procedure. This induces phase separation and formation of a polar phase and a separate organic phase, the organic phase comprising the halogenated alkoxyethane. In some embodiments, said organic phase may be separated from the polar phase before further processing. Separation may be effected according to any procedure known to a skilled person which would be fit for the intended purpose. For example, separation may be effected by means of the kind described herein. In these instances, the separated polar phase is discarded. The separated organic phase may undergo drying before being processed further. For example, the separated organic phase may be dried with a desiccant. Examples of suitable desiccants in that regard include inorganic desiccants such as magnesium sulfate.

Accordingly, in some embodiments of the purification procedure, the organic phase separated from the polar phase following addition of a polar liquid to the mixture obtained in step c) is dried with a desiccant before further processing. The desiccant may be magnesium sulfate.

In some embodiments, the purification procedure further comprises a step d) of isolating the purified halogenated alkoxyethane. The step may be performed on a dried organic phase obtained from the mixture obtained in step c) in accordance to a phase separation procedure of the kind described herein.

In step d) of the purification procedure, the purified halogenated alkoxyethane may be isolated by any suitable means known to a skilled person that would result in halogenated alkoxyethane with purity of at least 95%, for example at least 99%, such as about 99.9%. For example, in step d) of the purification procedure the purified halogenated alkoxyethane may be isolated by distillation. A skilled person would be able to readily identify suitable distillation conditions affording isolation of the halogenated alkoxyethane, for example based on the physical characteristics of the specific halogenated alkoxyethane and the nature and amount of any residual impurities.

In some embodiments, isolation of the purified halogenated alkoxyethane in step d) of the purification procedure is performed by fractional distillation. Those embodiments are particularly advantageous for the isolation of purified methoxyflurane obtained by reacting Cl₂C═CF₂ with a base of the kind described herein and methanol.

A skilled person would be able to readily identify suitable distillation conditions. For instance, fractional distillation in step d) of the purification procedure may be performed at a temperature above the boiling point of the halogenated alkoxyethane. In some embodiments, the distillation is performed at a temperature above 100° C.

Accordingly, in some embodiments the purification procedure comprises the steps of:
i. adding an amine to the reactor effluent or an organic phase separated from the reactor effluent,
ii. adding a polar liquid to the mixture obtained in step i) to induce phase separation and formation of a polar phase and a separate organic phase, the organic phase containing the halogenated alkoxyethane,
iii. adding an acid to the organic phase obtained in step ii), and
iv. isolating the purified halogenated alkoxyethane.

In some alternative embodiments, the purification procedure comprises the steps of:
i. adding an acid to the reactor effluent or an organic phase separated from the reactor effluent,
ii. adding a polar liquid to the mixture obtained in step i) to induce phase separation and formation of a polar phase and a separate organic phase, the organic phase containing the halogenated alkoxyethane,
iii. adding an amine to the organic phase obtained in step ii), and
iv. isolating the purified halogenated alkoxyethane.

In some embodiments, the purification procedure comprises a sequence of steps of the kind described herein. Accordingly, in some embodiments the distillation procedure comprises the steps of:
i. adding a polar liquid to the reactor effluent comprising the halogenated alkoxyethane to induce phase separation and formation of a polar phase and a separate organic phase comprising the halogenated alkoxyethane,
ii. separating the organic phase obtained in step i),
iii. adding one of the amine and the acid to the organic phase obtained in step ii),
iv. adding a polar liquid to the mixture obtained in step iii) to induce phase separation and formation of a polar phase and a separate organic phase, the organic phase containing the halogenated alkoxyethane,
v. separating the organic phase obtained in step iv),
vi. adding the other of the amine and the acid not used in step iii) to the organic phase obtained in step v),
vii. adding a polar liquid to the mixture obtained in step vi) to induce phase separation and formation of a polar phase and a separate organic phase, the organic phase containing the halogenated alkoxyethane,
viii. separating the organic phase obtained in step vii),
ix. drying the organic phase obtained in step viii), and
x. distilling the organic phase obtained in step ix) by fractional distillation, thereby isolating the purified halogenated alkoxyethane.

It will be understood that all compounds and procedural conditions of steps i)-x) listed in the preceding paragraph are compounds and procedural conditions of the kind described herein. Embodiments of the purification procedure having a sequence of said steps i)-x) are particularly advantageous for the purification of methoxyflurane obtained by reacting Cl₂C═CF₂ with a base of the kind described herein and methanol.

Specific embodiments of the invention will now be described with reference to the following non-limiting examples.

### EXAMPLES

### Example 1

A batch solution of potassium hydroxide (2.5% w/v) in methanol (1000 ml) was prepared and chilled in ice, and used as “material 1”. 1,1-Dichloro-2,2-difluoroethylene (DCDFE, 200 ml) was used as “material 2”.

A commercial plate reactor with five fluidic modules connected in series was used, providing for 45 ml of total reaction volume. The plate reactor could be purchased from any one of these available commercially such as AFR reactors from Corning, or glass or ceramic reactors from Chemtrix.

Material 1 was introduced into an inlet of the first fluidic module, at a flow rate of 10 ml/min, and Material 2 was introduced into a separate inlet of the fluidic plate at a flow rate of 2 ml/min. The temperature of the fluidic modules was controlled at 10° C.

A total volume of 80 ml of DCDFE was passed through the reactor, operated at steady state to provide a residence time of the reaction mixture of 3.75 minutes. The effluent was collected in fractions and the product obtained after separation as a clear colourless liquid after drying. Final combined volume=80 m (113.6 g, molar yield=80%, purity 97%).

Scheme 4 below shows the postulated mechanisms involving formation of impurities through further reactions of methoxyflurane.

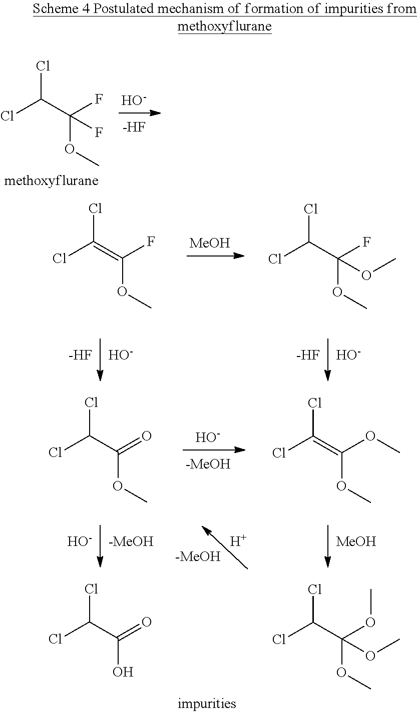

Impurities from the organic phase obtained by the procedure of Example 1 were mainly made by methoxyethene (ME) impurity.

Gas Chromatography (not shown) confirmed formation of methoxyflurane. The composition of subsequent fractions of the separated product are reported in Table 1 below. All fractions contained above 96.9% methoxyflurane (MEOF), with traces of chloroform, orthoester (OE), and methoxyethene (ME) impurities, as well as fractions of unreacted methanol and DCDFE. The level of purity was superior to that obtainable under batch reaction conditions, which is normally characterised by a reaction product at about 65% purity.

TABLE 1
composition of fractions of separated product

| | Methanol | DCDFE | Chloroform | MEOF | ME | TCTFCB | OE | Unknown |
|---|---|---|---|---|---|---|---|---|
| Fraction 1 | 1.800 | 0.388 | 0.004 | 97.158 | 0.372 | 0.025 | 0.030 | 0.163 |
| Fraction 2 | 1.834 | 0.413 | 0.004 | 97.048 | 0.397 | 0.026 | 0.033 | 0.186 |
| Fraction 3 | 1.981 | 0.477 | 0.004 | 96.846 | 0.406 | 0.025 | 0.026 | 0.169 |
| Fraction 4 | 1.897 | 0.485 | 0.004 | 96.913 | 0.415 | 0.025 | 0.023 | 0.168 |
| Fraction 5 | 1.965 | 0.477 | 0.004 | 96.855 | 0.416 | 0.025 | 0.026 | 0.167 |
| Fraction 6 | 1.779 | 0.394 | 0.004 | 97.108 | 0.419 | 0.026 | 0.028 | 0.178 |
| CF MDI | 0.021 | 0.146 | 0.011 | 98.785 | 0.641 | ND | — | — |

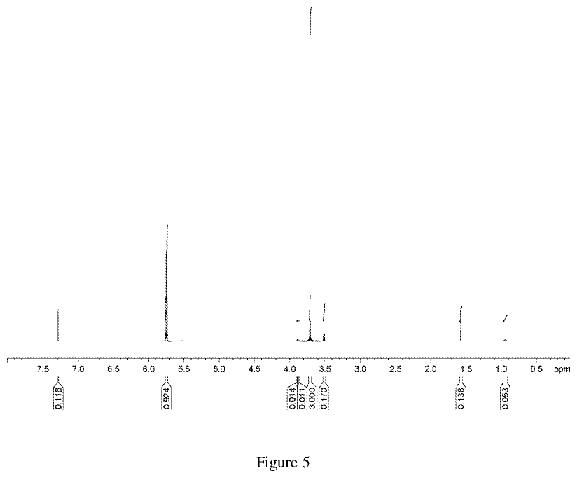

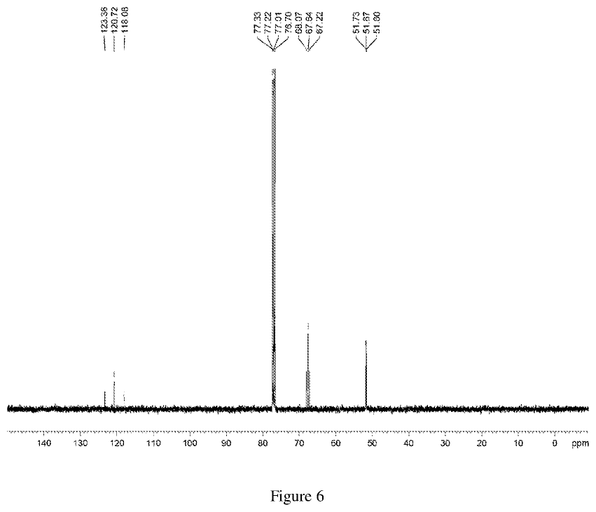

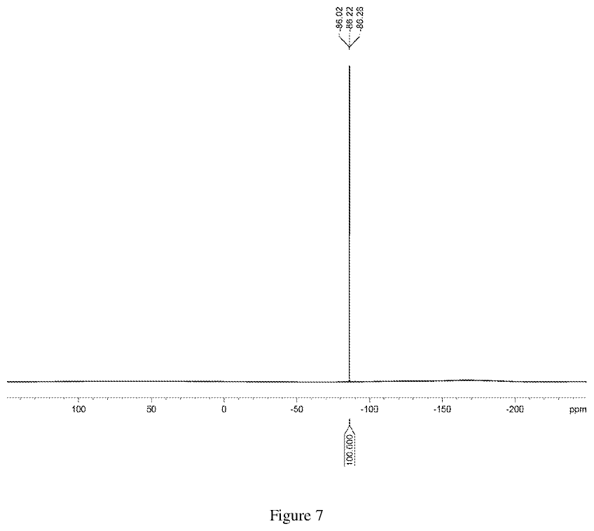

NMR was also performed on Fraction 4 listed in the Table, providing results shown in FIGS. 5-7. FIG. 5 relates to the ¹H trace, FIG. 6 relates to the ¹³C trace, and FIG. 7 relates to the ¹⁹F trace.

### Example 2

### Purification Process

### Removal of Methoxyethene (ME) and Orthoester (OE) Process Impurities.

Approximately 77 ml (110 g) of crude reaction product from Example 1 was transferred to a 3N 250-ml RBF fitted with a magnetic stirring device and temperature thermometer at ambient temperature (recorded at 20° C.). 9.5 ml of methane sulphonic acid was slowly added to the mixture over approximately 1 minute with stirring. The resulting mixture was left to stir for 120 minutes, where at this time 15 ml of water was added to the stirred mixture which was then allowed to stir for a further 60 minutes. The suspension was transferred to a separating funnel whereby the organic layer was removed from the aqueous layer. The organic layer was transferred back to the 3N 250 ml RBF and labelled CRUDE B.

### Removal of Methyl Dichloroacetate (MDA)

7.7 ml of ethanolamine was slowly added to the CRUDE B mixture over approx. 1 minute with stirring and ambient temperature. The resulting mixture was left to stir for approx. 30 minutes, whereby at this time 25 ml of water was added and the stirring stopped to allow the suspension phase separate. This suspension was transferred to a separation funnel and the organic layer removed from the aqueous layer. The organic phase was dried with magnesium sulphate and sampled for Purity. The resulting mixture (CRUDE C) had final volume of 71 ml (100.8 g, molar yield=70%, purity>99%, up to 99.9%).

As used herein, the term “about” when referring to a numeric value can encompass variations of, and in some embodiments, ±20%, in some embodiments ±10%, in some embodiments ±5%, in some embodiments ±1%, in some embodiments ±0.5%, and in some embodiments ±0.1%, from the specified numeric value.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Many modifications will be apparent to those skilled in the art without departing from the scope of the present invention.

The claims defining the invention are as follows:

1. A process for continuous preparation of halogenated alkoxyethane of general formula XClHC—CF₂OR, where X is —Cl or —F and OR is C₁₋₄ alkoxy, the process comprising a step of introducing in a plate reactor reaction components comprising (i) a compound of general formula XClC═CF₂, (ii) a base, and (iii) a C₁₋₄ alkanol, wherein
i. the plate reactor comprises a fluidic module defining one or more fluidic path(s) through which the reaction components flow as a reaction mixture, and
ii. the halogenated alkoxyethane is formed at least upon the reaction components mixing, with the so formed halogenated alkoxyethane flowing out of the plate reactor in a reactor effluent.

2. The process of claim 1, wherein the plate reactor comprises a stack of fluidic modules defining the one or more fluidic path(s) through which the reaction components flow as a reaction mixture.

3. The process of claim 1 or 2, wherein the fluidic module(s) is/are at a temperature of from about −15° C. to about 45° C.

4. The process of any one of claims 1-3, wherein the one or more fluidic path(s) provide a total internal volume of at least 10 ml.

5. The process of any one of claims 1-4, wherein the reactor effluent contains at least 90% by volume of the halogenated alkoxyethane.

6. The process of any one of claims 1-5, wherein the plate reactor provides a residence time of less than about 5 minutes.

7. The process of any one of claims 1-6, wherein the reaction mixture is obtained by mixing (i) a solution of the C₁₋₄ alkanol and the base with (ii) the compound of general formula XClC═CF₂.

8. The process of any one of claims 1-7, wherein the mixing is performed upstream of the one or more fluidic path(s).

9. The process of any one of claims 1-8, wherein the mixing is performed by combining (i) a flow of the solution of the C₁₋₄ alkanol and the base with (ii) a flow of the compound of general formula XClC═CF₂ according to a flow-rate ratio from 1:1 to 10:1.

10. The process of any one of claims 1-9, wherein the base is used in an amount of between 1% and 30% by weight relative to the total weight of base and C₁₋₄ alkanol.

11. The process of any one of claims 1-10, wherein the compound of general formula XClC═CF₂ is Cl₂C═CF₂ or FClC═CF₂.

12. The process of any one of claims 1-11, wherein the C₁₋₄ alkanol is selected from methanol (CH₃OH), ethanol (CH₃CH₂OH), 1-propanol (CH₃CH₂CH₂OH), 2-propanol ((CH₃)₂CHOH), 1-butanol (CH₃CH₂CH₂CH₂OH), 2-butanol (CH₃CH₂CHOHCH₃), 2-methyl-1-propanol ((CH₃)₂CHCH₂OH), 2-methyl-2-propanol ((CH₃)₃COH), and a combination thereof.

13. The process of any one of claims 1-12, wherein the halogenated alkoxyethane is Cl₂HC—CF₂OCH₃ (methoxyflurane) or ClFHC—CF₂OCH₃.

14. The process of any one of claims 1-13, wherein the base comprises an alkali metal base cation or an ammonium base cation.

15. The process of any one of claims 1-14, wherein the base is selected from sodium hydroxide, potassium hydroxide, sodium methoxide, potassium methoxide, tetrabutylammonium hydroxide, benzyl(trimethyl)ammonium hydroxide, N-methyl-N,N,N-trioctylammonium chloride, tetraethylammonium hydroxide, tetramethylammonium hydroxide, and tetramethylphosphonium hydroxide.

16. The process of any one of claims 1-15, further comprising a purification procedure that comprises the steps of:
a) adding one of an amine and an acid to the reactor effluent or an organic phase separated from the reactor effluent,
b) adding a polar liquid to the mixture obtained in step a) to induce phase separation and formation of a polar phase and a separate organic phase, the organic phase containing the halogenated alkoxyethane,
c) adding the other of the amine and the acid not used in step a) to the organic phase obtained in step b) to thereby purify the halogenated alkoxyethane.

17. The process of claim 16, further comprising a step d) of isolating the purified halogenated alkoxyethane.

18. The process of claim 16 or 17, wherein the amine is selected from ethylenediamine (1,2-diamnoethane), 1,3-diaminopropane, diethylenetriamine, di-n-propylamine, n-butylamine, ethanolamine, pyrrolidine, 2-aminobutane, and a mixture thereof.

19. The process of any one of claims 16-18, wherein the acid is selected from hydrochloric acid, sulfuric acid, sulphurous acid, methanesulfonic acid, trifluoromethanesulfonic acid, phosphoric acid, acetic acid, trifluoroacetic acid, nitric acid, nitrous acid, hypochlorous acid, chlorous acid, chloric acid, perchloric acid, and a combination thereof.

20. The process of any one of claims 16-19, wherein the purified halogenated alkoxyethane is isolated by fractional distillation.

21. The process of any one of claims 16-20, wherein the polar liquid is water.

22. The process of any one of claims 16-21, the process being for purifying the halogenated alkoxyethane from impurities comprising one or more of methoxyethene (ME), orthoesters (OE), and methyl dichloroacetate (MDA).

23. Halogenated alkoxyethane of general formula XClHC—CF₂OR, where X is —Cl or —F and OR is C₁₋₄ alkoxy, obtained in accordance with the process of any one of claims 16-22, the halogenated alkoxyethane having purity of at least 99%.
